(19) United States
(12) Patent Application Publication
(10) Pub. No.: US 2020/0281559 A1
(43) Pub. Date: Sep. 10, 2020

(54) ULTRASOUND ORAL CAVITY TOMOGRAPHY SYSTEM

(71) Applicant: Yuan-Ping HWANG, Hualien County (TW)

(72) Inventor: Yuan-Ping HWANG, Hualien County (TW)

(21) Appl. No.: 16/521,323

(22) Filed: Jul. 24, 2019

(30) Foreign Application Priority Data
Mar. 7, 2019 (TW) 108107622

(51) Int. Cl. A61B 8/08; A61B 5/00; A61B 8/00

(52) U.S. Cl. CPC A61B 8/08; A61B 5/4542; A61B 8/13; A61B 8/4444; A61B 8/4209

(57) ABSTRACT

The present invention provides an ultrasound oral cavity tomography system, comprising a chin-supporting structure, a plurality of U-shaped ultrasound detectors, and an ultrasound examination unit. The chin-supporting structure is provided with a recess. Each of the U-shaped ultrasound detectors comprises a plurality of ultrasound probes arranged in a U-shaped array. The ultrasound examination unit is connected to the U-shaped ultrasound detectors and configured to form three-dimensional images of an oral cavity based on signals received by the U-shaped ultrasound detectors.

### BACKGROUND OF THE INVENTION

### 1. Technical Field

The present invention relates to an ultrasound tomography system and more particularly to an ultrasound tomography system configured to facilitate medical diagnosis regarding the oral cavity.

### 2. Description of Related Art

With the rapid development of the medical industry, a variety of non-invasive, non-radioactive examination methods have found extensive clinical application. These methods are less harmful to the human body than those involving X-rays, computed tomography (CT), or magnetic resonance imaging (MRI).

Of the former type of examination methods, ultrasound medical examination is the most widely used. An ultrasound medical examination typically includes sending ultrasonic waves into the body tissues to be examined (e.g., muscles or organs), detecting ultrasonic echoes, and plotting the detected echoes to form images that visualize the sizes and structures of the muscles or organs. Ultrasound medical examination has occupied an irreplaceable position in medical diagnostics not only because it features non-radioactivity and hence great safety, but also because it provides real-time images and thereby saves the time otherwise required for film development or digital imaging. Medical ultrasound is commonly used in the obstetric flied to monitor and measure the growth of a fetus in real time. When applied to the cardiovascular system, medical ultrasound helps determine the velocity of blood flow so that the condition of a disease can be identified rapidly.

Many diseases show their early symptoms in the oral cavity. For instance, oral ulcers are common to those who have an autoimmune disease (AID) such as AIDS (acquired immune deficiency syndrome), lupus erythematosus, or herpetic dermatitis. In view of this, the inventor of the present invention thought it necessary to devise a medical diagnosis technique that applies medical ultrasound to the oral cavity, the goal being to expedite the diagnosis of oral lesions and treatment of the underlying diseases.

### BRIEF SUMMARY OF THE INVENTION

The primary objective of the present invention is to provide a system that scans the oral cavity with ultrasonic waves to assist in medical diagnosis.

In order to achieve the above objective, the present invention provides an ultrasound oral cavity imaging system, comprising a chin-supporting structure, a plurality of U-shaped ultrasound detectors, and an ultrasound examination unit. The chin-supporting structure is provided with a recess. Each of the U-shaped ultrasound detectors comprises a plurality of ultrasound probes arranged in a U-shaped array. The ultrasound examination unit is connected to the U-shaped ultrasound detectors and configured to form three-dimensional images of an oral cavity based on signals received by the U-shaped ultrasound detectors.

Furthermore, the chin-supporting structure comprises a plurality of tiers each defining a recess area, the recess is formed by the recess areas of the tiers, the recess areas of the tiers are gradually reduced toward a bottom side of the chin-supporting structure, and the U-shaped ultrasound detectors are provided respectively in the tiers of the chin-supporting structure.

Furthermore, each of the U-shaped ultrasound detectors defines a surrounded area, and the surrounded areas of the U-shaped ultrasound detectors are gradually reduced toward the bottom side of the chin-supporting structure to match the gradually reduced recess areas of the tiers.

Furthermore, the recess comprises a nose-accommodating recess formed correspondingly to each of the tiers.

Furthermore, the wall of the recess is coated with a wave guiding material.

Furthermore, the wave guiding material is a water-based gel.

Furthermore, the ultrasound examination unit renders the images obtained into colored or grayscale images according to the intensities of feedback signals.

Comparing to the conventional techniques, the present invention has the following advantages:

1. The system disclosed herein includes a chin-supporting structure on which a patient can rest his/her chin to facilitate three-dimensional (3D) imaging of the oral cavity. The disclosed system is adaptable to various face shapes and sizes and can therefore be used to examine patients over a wide range of ages.

2. The disclosed system includes a plurality of U-shaped ultrasound detectors to prevent insufficient imaging and reduce potential injury to the human body. Moreover, the disclosed system can directly output 3D images of the oral cavity without having to convert two-dimensional (2D) images into 3D ones in a subsequent step.

### BRIEF DESCRIPTION OF THE SEVERAL VIEWS OF THE DRAWINGS

FIG. 1 is a block diagram of the ultrasound oral cavity tomography system according to the first embodiment of the present invention.

FIG. 2 is a perspective view of the chin-supporting structure and the U-shaped ultrasound detectors according to the first embodiment of the present invention.

FIG. 3 is a sectional view of the chin-supporting structure and the U-shaped ultrasound detectors according to the first embodiment of the present invention.

FIG. 4 is a perspective view of the chin-supporting structure and the U-shaped ultrasound detectors in the second embodiment of the present invention.

FIG. 5 is a sectional view of the chin-supporting structure and the U-shaped ultrasound detectors in the second embodiment of the present invention.

### DETAILED DESCRIPTION OF THE INVENTION

The details and technical solution of the present invention are hereunder described with reference to accompanying drawings. For illustrative sake, the accompanying drawings are not drawn to scale. The accompanying drawings and the scale thereof are not restrictive of the present invention.

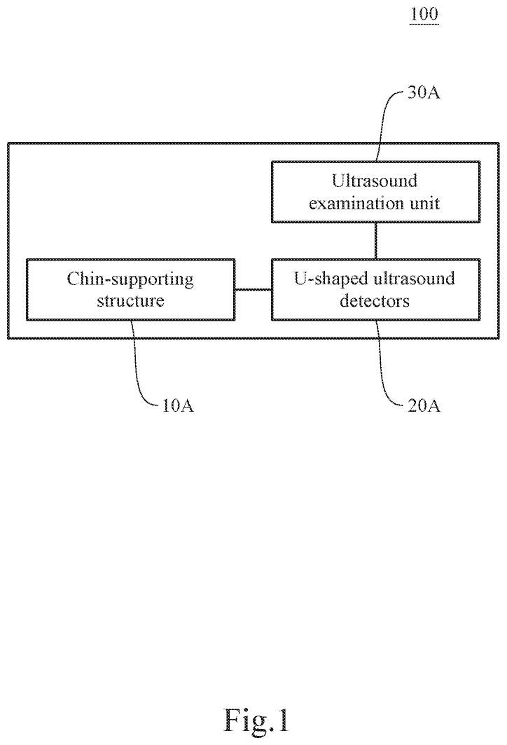

Please refer to FIG. 1 for a block diagram of the ultrasound oral cavity tomography system according to an embodiment of the present invention.

As shown in FIG. 1, the ultrasound oral cavity tomography system 100 includes a chin-supporting structure 10A, a plurality of U-shaped ultrasound detectors 20A, and an ultrasound examination unit 30A. The ultrasound oral cavity tomography system 100 is intended mainly for medical care of the oral cavity and, taking advantage of the ability of medical ultrasound to penetrate human muscles and soft tissues, is configured to scan the oral cavity with ultrasonic waves, construct 3D images of the oral cavity scanned, and analyze the 3D images to assist in pathological diagnosis and subsequent treatment.

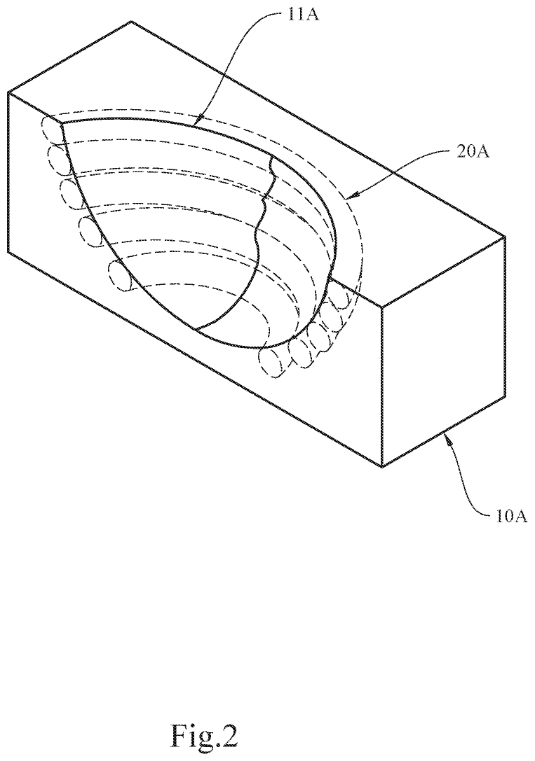

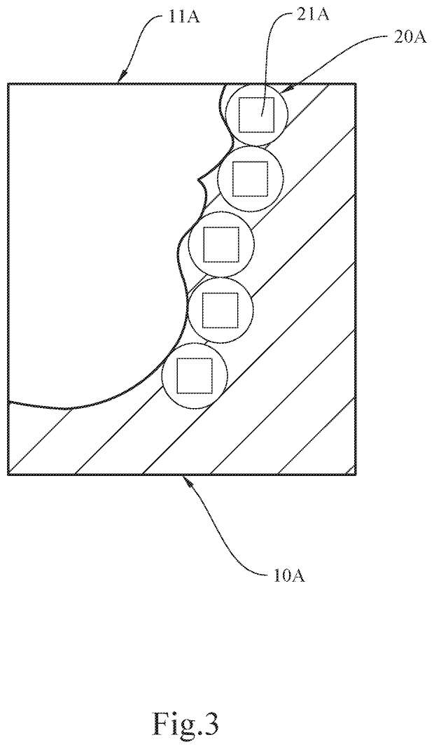

Please refer to FIG. 2 and FIG. 3 respectively for a perspective view and a sectional view of the chin-supporting structure 10A and the U-shaped ultrasound detectors 20A of the present invention.

As shown in FIG. 2 and FIG. 3, the chin-supporting structure 10A is provided with a recess 11A in order for a subject to rest their chin on the wall of the recess 11A. In one preferred embodiment, the recess 11A is further provided with a nose-accommodating recess corresponding in position to the subject's nose.

To produce preferred detection results, the wall of the recess 11A is coated with a wave guiding material for producing a coupling effect between the subject's skin and the U-shaped ultrasound detectors 20A so that ultrasonic waves can enter and exit the subject's skin with ease. In one preferred embodiment, the wave guiding material is a water-based gel.

Each U-shaped ultrasound detector 20A includes a plurality of ultrasound probes 21A, which are arranged in a U-shaped array and are provided behind and along the wall of the recess 11A. The U-shaped ultrasound detectors 20A are sequentially arranged, starting from an upper position in the chin-supporting structure 10A to a lower position in the chin-supporting structure 10A and in a way that conforms to the shape of the recess 11A so as to adapt to various face shapes and sizes and correspond precisely to the subject's oral cavity during examination.

In ultrasound medical examination, a phased array of piezoelectric transducers (generally made of ceramic) is typically used to generate short and strong acoustic pulses that form acoustic waves. Each ultrasound probe 21A, therefore, includes a piezoelectric transducer packaged therein along with the related wires, in order for the ceramic transducer to oscillate when supplied with electrical pulses and thereby generate a series of acoustic pulses. The frequency of the resulting acoustic waves may be any frequency in the range of 1 to 13 THz and is hence far higher than those audible to human ears. The term “ultrasonic waves” as used herein refers to any acoustic wave whose frequency is higher than those able to be heard by human beings. The acoustic waves of the transducers will combine into a single acoustic wave that is focused and arcuate. The higher the frequency, the shorter the corresponding wavelength; and the shorter the wavelength, the higher the resolution of the image obtained. However, as the speed at which acoustic waves attenuate increases with the frequency of the acoustic waves, a relatively low frequency (3 to 5 THz) is preferable in order to probe tissues that are deep in the human body.

Each ultrasound probe 21A is coated with rubber so that acoustic waves can propagate effectively into the subject (i.e., to achieve a match in impedance). The acoustic waves are partially reflected back to the probes by interfaces between different tissues, wherein the reflected acoustic waves are generally referred to as echoes. As is well known in the art, even tiny structures generate echoes (i.e., can reflect acoustic waves).

The paths taken by the echoes (i.e., the acoustic waves returning to, and to be received by, the ultrasound probes 21A) are similar to those of the acoustic waves emitted from the ultrasound probes 21A, except that the former paths and the latter paths run in opposite directions. The returning acoustic waves cause the transducers in the ultrasound probes 21A to oscillate, and the oscillation is converted into electrical pulses by the transducers. The ultrasound probes 21A send the electrical pulses to the ultrasound examination unit 30A in order for the ultrasound examination unit 30A to process the electrical pulses and thereby generate digital images.

The ultrasound examination unit 30A is connected to the U-shaped ultrasound detectors 20A and is configured to construct 3D images of the subject's oral cavity based on the signals received by the U-shaped ultrasound detectors 20A. More specifically, the ultrasound examination unit 30A is an image processing device for constructing 3D images according to the data fed back from each pixel (i.e., ultrasound probe 21A) of the U-shaped ultrasound detectors 20A. The ultrasound examination unit 30A receives three major types of parameters from the ultrasound probes 21A: the location of each ultrasound probe 21A that has received an echo (i.e., the location of each response-receiving pixel in an array), the signal intensity of each echo, and the flight times of ultrasonic waves (i.e., the response times).

Once the three types of data are obtained, the ultrasound examination unit 30A constructs a 3D model of the examined area according to the data. Construction of the 3D image model may include applying time-division multiplexing (TDM) to the responses to the plural ultrasound probes 21A. The coordinates of each response-receiving pixel (i.e., the relative coordinates or world coordinates of the pixel in a 3D space) can be derived from the location of the corresponding response-receiving ultrasound probe 21A and the corresponding flight time of ultrasonic waves. Moreover, in order to be mapped to a 3D space, the original images must be corrected in accordance with the locations of the ultrasound probes 21A while being converted into 3D images. For example, a reference point is set in the world coordinate system, and mapping computation is performed with reference to the reference point. Tissue densities in different areas can be derived from the signal intensities of the echoes and the flight times of ultrasonic waves, before the tissues are stratified depth-wise. The 3D images obtained can also be filtered using specific threshold values in order to produce images only of the area of interest, such as a dental caries, a stomatitis lesion (e.g., of the gum, an alveolar ridge, the periodontium, or the oral mucosa), an oral malformation (e.g., a dental malposition or cleft palate), an oral cancer, a cyst, or a fungal lesion. The depth to which ultrasonic waves penetrate the subject (i.e., the sampling depth) can be changed by adjusting the power and frequency of the ultrasonic waves so that an image model can be constructed for a relatively shallow or relatively deep area. In another preferred embodiment, the ultrasound examination unit 30A can render the images obtained into colored or grayscale images according to the intensities of feedback signals. More specifically, the images obtained can be filled with different grayscale values or colors by setting specific threshold values, in order to accentuate the images of individual tissues in the oral cavity.

Apart from the algorithms stated above, the present invention may use a single-input multi-output (SIMO) model, a multi-input single-output (MISO) model, or a multi-input multi-output (MIMO) model without limitation.

In this embodiment, the ultrasound examination unit 30A as well as the ultrasound probes 21A surrounding the target area to be examined is configured on the assumption that the speed of sound is constantly 1540 m/s. While the echoes may lose some of the acoustic energy of the original acoustic waves, the loss is nominal when compared with attenuation caused by absorption.

The following paragraphs disclose the ultrasound oral cavity tomography system according to the second preferred embodiment of the present invention. The second embodiment is different from the first embodiment in the structure of the chin-supporting structure. The remaining aspects of the second embodiment are identical to those of the first embodiment and therefore will not be described repeatedly.

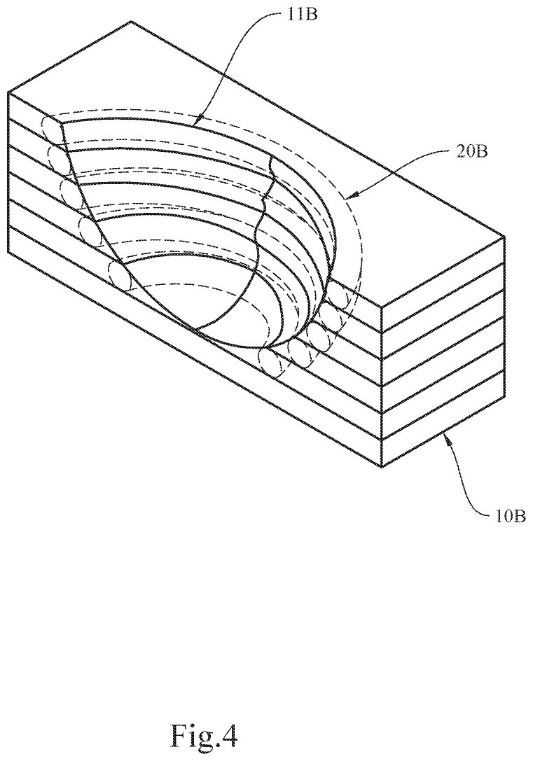

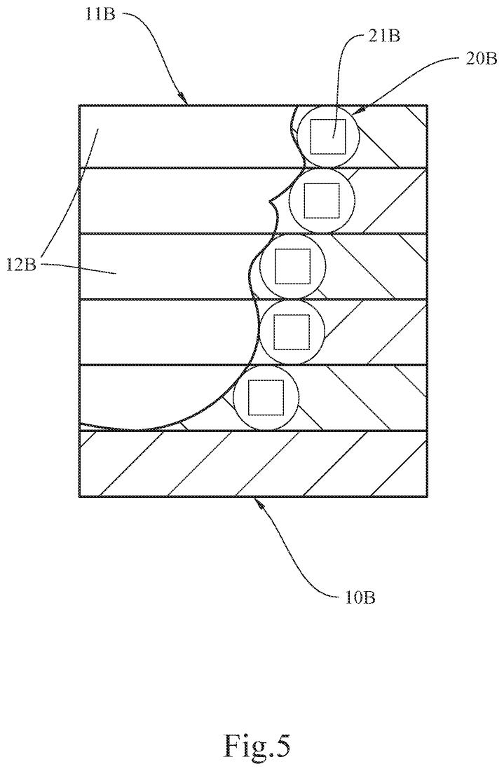

Please refer to FIG. 4 and FIG. 5 respectively for a perspective view and a sectional view of the chin-supporting structure and the U-shaped ultrasound detectors in the second embodiment of the present invention.

In this embodiment, the chin-supporting structure 10B is provided with a recess 11B in order for a subject to rest their chin on the wall of the recess 11B. The chin-supporting structure 10B includes a plurality of tiers 12B, and each tier 12B defines a recess area. The recess areas of the tiers 12B are gradually reduced toward the bottom side of the chin-supporting structure 10B and jointly form the recess 11B. To use, a selected number of tiers 12B (whose recess areas have different widths and depths) are put together according to the subject's face shape and size. The chin-supporting structure 10B, therefore, can be used more flexibly than its counterpart in the previous embodiment. While the recess 11B in FIG. 4 is defined by five tiers 12B vertically stacked together, the present invention has no limitation on the number of the tiers 12B. For example, there may be four, six, or another number of tiers 12B in another embodiment of the present invention. The recess 11B may include a nose-accommodating recess formed correspondingly to each of the tiers 12B and corresponding in position to the subject's nose.

The U-shaped ultrasound detectors 20B are provided respectively in the tiers 12B of the chin-supporting structure 10B. Each U-shaped ultrasound detector 20B includes a plurality of ultrasound probes 21B, which are arranged in a U-shaped array and are provided behind and along the wall of the recess 11B. Each U-shaped ultrasound detector 20B surrounds and thereby defines a surrounded area. The surrounded areas of the U-shaped ultrasound detectors 20B are gradually reduced toward the bottom side of the chin-supporting structure 10B to match the gradually reduced recess areas of the tiers 12B, in order for the U-shaped ultrasound detectors 20B to adapt to various face shapes and sizes and correspond precisely to the subject's oral cavity during examination.

According to the above, the present invention provides a chin-supporting structure provided with a plurality of U-shaped ultrasound detectors. A patient can rest his/her chin on the disclosed system to facilitate three-dimensional (3D) imaging of the oral cavity. The disclosed system is adaptable to various face shapes and sizes and can therefore be used to examine patients over a wide range of ages. Meanwhile, the disclosed system can not only prevent insufficient imaging and reduce potential injury to the human body but also directly output 3D images of the oral cavity without having to convert two-dimensional (2D) images into 3D ones in a subsequent step.

The above is the detailed description of the present invention. However, the above is merely the preferred embodiment of the present invention and cannot be the limitation to the implement scope of the present invention, which means the variation and modification according to the present invention may still fall into the scope of the invention.

What is claimed is:

1. An ultrasound oral cavity tomography system, comprising:
a chin-supporting structure provided with a recess;
a plurality of U-shaped ultrasound detectors, each comprising a plurality of ultrasound probes arranged in a U-shaped array; and
an ultrasound examination unit connected to the U-shaped ultrasound detectors and configured to form three-dimensional images of an oral cavity based on signals received by the U-shaped ultrasound detectors.

2. The ultrasound oral cavity tomography system of claim 1, wherein the chin-supporting structure comprises a plurality of tiers each defining a recess area, the recess is formed by the recess areas of the tiers, the recess areas of the tiers are gradually reduced toward a bottom side of the chin-supporting structure, and the U-shaped ultrasound detectors are provided respectively in the tiers of the chin-supporting structure.

3. The ultrasound oral cavity tomography system of claim 2, wherein each of the U-shaped ultrasound detectors defines a surrounded area, and the surrounded areas of the U-shaped ultrasound detectors are gradually reduced toward the bottom side of the chin-supporting structure to match the gradually reduced recess areas of the tiers.

4. The ultrasound oral cavity tomography system of claim 2, wherein the recess comprises a nose-accommodating recess formed correspondingly to each of the tiers.

5. The ultrasound oral cavity tomography system of claim 1, wherein the wall of the recess is coated with a wave guiding material.

6. The ultrasound oral cavity tomography system of claim 5, wherein the wave guiding material is a water-based gel.

7. The ultrasound oral cavity tomography system of claim 1, wherein the ultrasound examination unit renders the images obtained into colored or grayscale images according to the intensities of feedback signals.
